(19) United States
(12) Patent Application Publication
(10) Pub. No.: US 2011/0237941 A1
(43) Pub. Date: Sep. 29, 2011

(54) DIRECTIONAL RADIATION DETECTOR

(75) Inventors: Arie Shahar, Moshav Magshimim (IL); Uri El-Hanany, Rehovot (IL); Zeev Gutman, Kfar Mordehai (IL); Shimon Cohen, Ness Ziona (IL); Dan Zemer, Rehovot (IL); Aharon Amrami, Yokneam (IL)

(73) Assignee: Orbotech Ltd., Yavne (IL)

(21) Appl. No.: 12/599,370

(22) PCT Filed: May 6, 2008
(86) PCT No.: PCT/IL08/00620

§ 371 Date: Jul. 15, 2010

Related U.S. Application Data
(63) Continuation-in-part of application No. 11/801,084, filed on May 8, 2007, now abandoned.

(51) Int. Cl. A61B 6/03; G01T 1/00; A61B 6/00

(52) U.S. Cl. 600/427; 378/4; 250/394; 600/436

(57) ABSTRACT

A method for imaging a body, including scanning the body so as to generate a tomographic image thereof, and analyzing the tomographic image to determine a location of a region of interest (ROI) (38) within the body. The method includes providing single photon counting detector modules (40), each of the modules being configured to receive photons from a respective direction and to generate a signal in response thereto. The method further includes coupling each of the modules to a respective adjustable mount (54), adjusting each of the adjustable mounts so that the direction of the module coupled thereto is aligned with respect to the location so as to receive radiation from the ROI, operating each of the modules to receive the photons from the ROI, and, in response to the signal generated by each of the modules, generating a single photon counting image of the ROI.

### CROSS-REFERENCE TO RELATED APPLICATION

This application is related to the U.S. patent application titled “Variable Collimation in Radiation Detection,” filed 28 Mar. 2007, which is assigned to the assignee of the present invention and which is incorporated herein by reference.

### FIELD OF THE INVENTION

The present invention relates generally to imaging, and specifically to medical imaging using multiple types of radiation and multiple imaging methods and systems.

### BACKGROUND OF THE INVENTION

A number of methods are known for non-invasively imaging internal organs, or characteristics of organs, of a patient. Such methods include X-ray and magnetic resonance imaging which use emitted radiation which behaves, and may typically be treated, largely according to its wave properties. The methods also include analyzing of radiation caused by a radioisotope that is injected into the patient. The radiation emitted in these cases may be direct or indirect emission of γ-rays. Direct emission of γ-rays may be from decay of a radioisotope such as ^{99m}Tc. Indirect γ-ray emission may be generated by annihilation of positrons produced by a positron emitter such as ¹⁸F. Both types of γ-ray emissions behave largely according to particle properties, and are usually termed single photon emissions.

Images produced by the emission systems described above may be enhanced by generating multiple images. The multiple images may be processed, by computerized tomography (CT), to give derived images which depict the internal organs and their characteristics in greater detail than the unenhanced images. However, all image producing methods have advantages and disadvantages.

### SUMMARY OF THE INVENTION

In embodiments of the present invention, the body of a patient is scanned sequentially by two imaging processes. A first process determines a location of a particular region of interest (ROI) in the body. A second process images the ROI using a single photon emission computerized tomography (SPECT) system. By first determining the location of the ROI, then imaging the ROI with the SPECT system, the overall time required for high quality SPECT imaging of the ROI is significantly reduced compared to the time required for producing SPECT images having the same high quality if the ROI is not first located.

In some embodiments, the two processes are performed by two separate imaging systems. For example, a first imaging system comprises a computerized tomograph (CT), typically an X-ray CT. A processor operates the CT to produce multiple CT images of the body. The multiple CT images are analyzed by the processor automatically. In some cases the analysis may be performed with the help of an operator of the CT. The analysis determines position coordinates of the ROI, as well as view angles of the ROI from multiple positions around the ROI.

A second, SPECT, imaging system comprises a plurality of single photon counting detector modules, each single photon counting detector module being coupled to a respective adjustable mount. The SPECT system includes sensors that provide the position coordinates of each single photon counting detector module. The coordinates of the ROI are derived by the processor using the information acquired by the CT system. Using the information of the coordinates of the single photon counting detector modules and the ROI, the processor aligns the mounts so that their coupled single photon counting detector modules are directed towards the ROI. The processor then operates the single photon counting detector modules to receive photons from the ROI. Using signals from the modules, the processor generates a single photon emission counting tomography image of the ROI. The method for generating the image may be applied regardless of whether the SPECT system operates in a mobile, typically a rotational, mode or in a static mode.

The SPECT system may be a stand alone system or a subsystem in an integrated CT-SPECT system.

In some embodiments, prior to using the SPECT system, an operator of the system produces computer simulations of images generated by the system. The computer simulations are typically produced for a variety of organs or other ROIs, such as the heart, the liver, or a kidney. For the simulations, each organ/ROI is assumed to have received a single photon emitter, typically a radio-isotope. During a specific simulation, parameters of each single photon detector, such as its dimensions, orientation and location with respect to the organ/ROI, an acquisition time for the detector at the location and with the orientation, and properties of the collimator coupled to the detector, are varied. The parameters are set within limits that apply for the actual detectors, according to an operator-set scanning strategy for the detectors.

For each organ/ROI, a group of such simulations is prepared, each simulation generating a respective simulated image of the organ/ROI. The images are analyzed to determine a best image. Detector parameters used to generate the best image are saved in an optimal detector scanning strategy, for use in performing the actual imaging of the organ/ROI. Typically, an optimal detector scanning strategy is generated for each organ/ROI. In addition, optimal strategies may be generated for a specific organ/ROI having differing characteristics, such as livers having different dimensions, or hearts at different gated times during the beat cycle. In some embodiments the detector parameters of the optimal scanning strategy are selected so as to have the motion of one or more of the detectors be in a single direction, rather than in a sweep or back-and-forth motion.

To operate the SPECT system, typically an operator of the system chooses an appropriate optimal detector scanning strategy from those generated by the simulations. Alternatively, a processor in the system may be configured to automatically choose the optimal scanning strategy according to parameters preset by the operator. For example, the operator may configure the processor to choose as the optimal strategy the strategy having a shortest overall scan time. The strategy chosen depends upon the organ/ROI being scanned, as well as upon characteristics of the organ/ROI, both of which may be determined from the first process.

In alternative embodiments, the two processes are performed by the SPECT imaging system operating in two configurations, and no other imaging system is required. The two configurations are implemented by coupling an adjustable collimating system to each of the single photon counting detector modules. The ROI may be located with the collimating systems adjusted to have a relatively large solid angle of acceptance, thereby generating a coarse quality image quickly. The final image may be generated with the collimating systems adjusted to have a relatively small solid angle of acceptance, and by realigning, if necessary, the single photon counting detector modules. The module realignment may be performed by a processor according to the coordinates of the ROI, derived from the coarse quality image, and from the coordinates of the modules measured, inter alia, by the module position sensors. The final image thus has a fine quality, and may be generated in a relatively short time.

There is therefore provided, according to an embodiment of the present invention, a method for imaging a body, including:

scanning the body so as to generate a tomographic image thereof;

analyzing the tomographic image to determine a location of a region of interest (ROI) within the body;

providing a plurality of single photon counting detector modules, each of the single photon counting detector modules being configured to receive photons from a respective direction and to generate a signal in response thereto;

coupling each of the single photon counting detector modules to a respective adjustable mount;

adjusting each of the adjustable mounts so that the direction of the single photon counting detector module coupled thereto is aligned with respect to the location so as to receive radiation from the ROI;

operating each of the single photon counting detector modules to receive the photons from the ROI; and

in response to the signal generated by each of the single photon counting detector modules, generating a single photon counting image of the ROI.

Typically, scanning the body includes scanning the body with an imaging system other than the plurality of single photon counting detector modules, and the imaging system may include a computerized tomography imaging system.

In an embodiment each of the adjustable mounts is individually adjustable, and adjusting the adjustable mounts includes adjusting the mounts independently of each other.

In an alternative embodiment adjusting each of the adjustable mounts includes adjusting a distance of at least one of the modules from a surface of the body to be within a preset range. Typically, the preset range is between of the order of 1 cm and 0 cm.

In a further alternative embodiment adjusting each of the adjustable mounts includes measuring a location coordinate and/or an orientation of at least one of the modules.

The plurality of the single photon counting detector modules may be configurable in a multiplicity of system configurations wherein the modules receive the radiation from a multiplicity of respective different volumes enclosing the ROI. Typically, scanning the body includes arranging the plurality of the single photon counting detector modules in a first of the multiplicity to have a first volume enclosing the ROI, and adjusting each of the adjustable mounts includes arranging the plurality of the single photon counting detector modules in a second of the multiplicity to have a second volume enclosing the ROI and smaller than the first volume.

In a disclosed embodiment at least one of the single photon counting detector modules is operative in a first unit configuration wherein the at least one module is arranged to receive radiation from a first solid angle, and is operative in a second unit configuration wherein the at least one module is arranged to receive radiation from a second solid angle different from the first solid angle.

In an alternative disclosed embodiment operating each of the single photon counting detector modules includes operating the single photon counting detector modules in an operating mode selected from a group of modes including a rotational mode and a static mode.

Typically, the single photon counting image of the ROI includes a single photon emission computerized tomography (SPECT) image.

There is further provided, according to an embodiment of the present invention, apparatus for imaging a body, including:

a plurality of single photon counting detector modules, each of the single photon counting detector modules being configured to receive photons from a respective direction and to generate a signal in response thereto;

a plurality of adjustable mounts respectively coupled to the single photon counting detector modules; and

a processor which is configured to analyze a tomographic image so as to determine a location of a region of interest (ROI) within the body, to adjust each of the adjustable mounts so that the direction of the single photon counting detector module coupled thereto is aligned with respect to the location so as to receive radiation from the ROI, to operate each of the single photon counting detector modules to receive the photons from the ROI, and in response to the signal generated by each of the single photon counting detector modules, to generate a single photon counting image of the ROI.

The apparatus may include an imaging system, other than the plurality of single photon counting detector modules, which is configured to generate the tomographic image.

There is further provided, according to an embodiment of the present invention, apparatus for imaging a region of interest (ROI) within a body having an outer surface, including:

a single photon counting detector module including:

a two-dimensional array of photon counting detectors, each of the detectors being configured to generate a signal indicative of a radio-isotope concentration in the ROI in response to a respective flux of photons received from the radio-isotope concentration; and

a plurality of collimator channels respectively coupled and aligned with the photon counting detectors in the two-dimensional array so that each of the photon counting detectors is able to receive the respective flux of the photons via its coupled collimator channel, the plurality of collimator channels being connected together so as to form a module outer surface; and

an adjustable mount to which the module is fixedly connected and which is configured to set an orientation of the module with respect to the ROI and to set a location of the module outer surface with respect to the outer surface of the body so that all of the photon counting detectors are able to simultaneously receive from the ROI the respective flux of the photons.

There is further provided, according to an embodiment of the present invention, a method for imaging a region of interest (ROI) within a body having an outer surface, including:

providing a single photon counting detector module comprising a two-dimensional array of photon counting detectors, each of the detectors being configured to generate a signal indicative of a radio-isotope concentration in the ROI in response to a respective flux of photons received from the radio-isotope concentration;

coupling and aligning a plurality of collimator channels respectively with the photon counting detectors in the two-dimensional array so that each of the photon counting detectors is able to receive the respective flux of the photons via its coupled collimator channel;

connecting the plurality of collimator channels together so as to form a module outer surface;

fixedly connecting an adjustable mount to the module; and

configuring the mount to set an orientation of the module with respect to the ROI and to set a location of the module outer surface with respect to the outer surface of the body so that all of the photon counting detectors are able to simultaneously receive from the ROI the respective flux of the photons.

The present invention will be more fully understood from the following detailed description of the embodiments thereof, taken together with the drawings, a brief description of which follows.

### BRIEF DESCRIPTION OF THE DRAWINGS

FIG. 1 is a schematic diagram of an imaging facility, according to an embodiment of the present invention;

FIG. 2 is a schematic diagram of a photon imaging unit, according to an embodiment of the present invention;

FIG. 3 is a schematic diagram showing a SPECT imaging system in relation to a patient and a region of interest in the patient, according to an embodiment of the present invention;

FIG. 4 is a flowchart of a process used by a processor in the imaging facility of FIG. 1, according to an embodiment of the present invention

FIG. 5 is a schematic diagram of an alternative photon imaging unit, according to an embodiment of the present invention;

FIG. 6 is a schematic diagram illustrating an alternative SPECT imaging system in relation to a patient and a region of interest in the patient, according to an embodiment of the present invention;

FIG. 7 is a schematic diagram of an alternative imaging facility, according to an embodiment of the present invention;

FIG. 8 is a flowchart of an alternative process used by a processor in the facility of FIG. 7, according to an embodiment of the present invention;

FIG. 9 is a flowchart of a simulation process that may be applied in the facilities of FIG. 1 or FIG. 7, according to an embodiment of the present invention;

FIGS. 10A and 10B show diagrams illustrating the process of FIG. 9, according to an embodiment of the present invention;

FIGS. 11A and 11B illustrate a simulated image, and a schematic corresponding setup in the facility of FIG. 1, according to an embodiment of the present invention;

FIG. 12 is a flowchart of an alternative process used by the processor in the facility of FIG. 1, according to an embodiment of the present invention;

FIG. 13 is a flowchart of a disclosed process used by the processor in the facility of FIG. 7, according to an embodiment of the present invention; and

FIG. 14 is a flowchart of a another alternative process used by the processor in the facility of FIG. 1, according to an embodiment of the present invention.

### DETAILED DESCRIPTION OF EMBODIMENTS

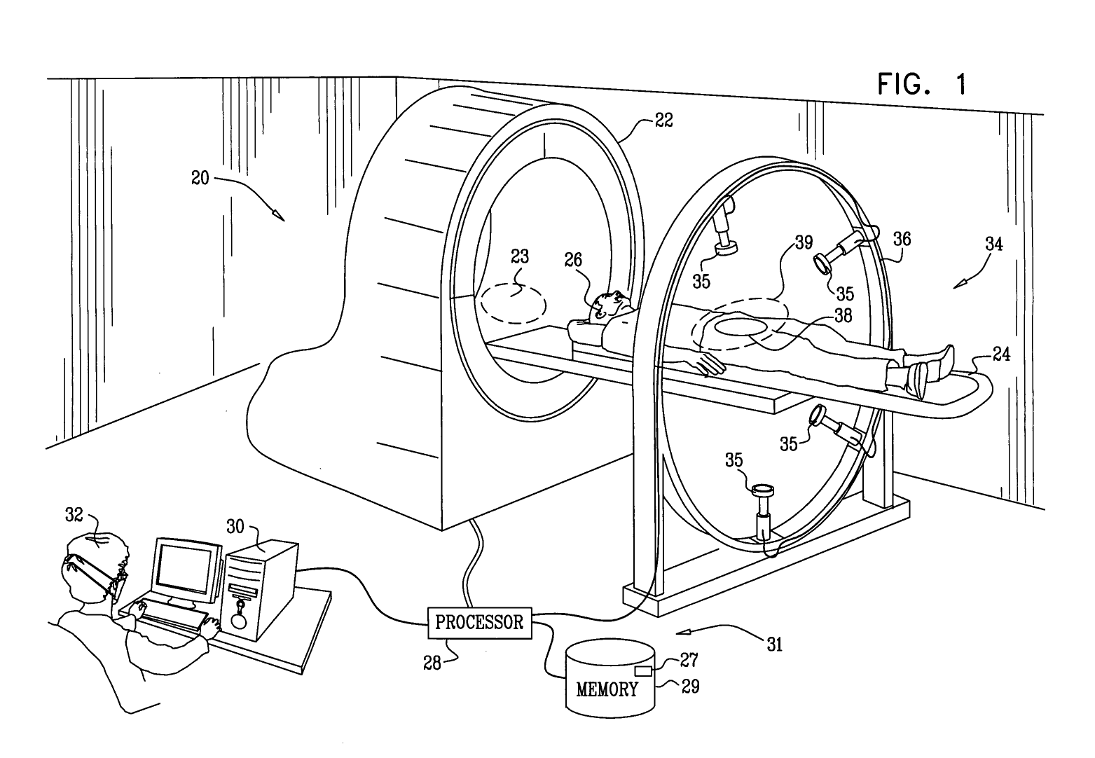

Reference is now made to FIG. 1, which is a schematic diagram of an imaging facility 20, according to an embodiment of the present invention. Facility 20 uses two systems for imaging a patient 26: a single photon emission computerized tomography (SPECT) imaging system 34, herein also termed primary imaging system 34, and a secondary imaging system 22. Secondary imaging system 22 may be used to locate a region of interest (ROI) 38 of a patient 26 that is to be imaged by the primary imaging system.

Secondary imaging system 22 typically comprises a computerized tomography (CT) machine such as an X-ray CT machine. However, embodiments of the present invention may use CT machines other than X-ray CT machines, such as CT machines that use magnetic resonance imaging (MRI). Furthermore, embodiments of the present invention may use other types of secondary imaging system, such as an ultrasonic array, for locating ROI 38. Other modalities for locating ROI 38 include, but are not limited to, electroencephalography (EEG), electrocardiography (ECG), EEG and ECG imaging (EEGI and ECGI), and measurements of one or more physiological variables, for example sound and/or temperature measurements, on organs such as a beating heart.

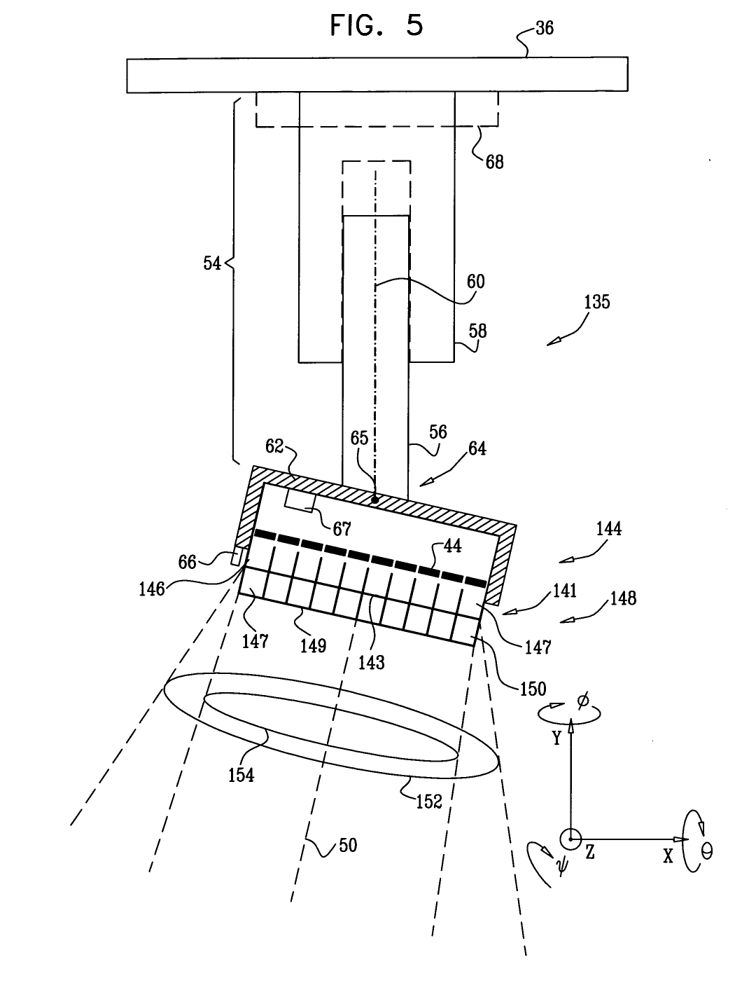

In some embodiments of the present invention, described in more detail below with respect to FIGS. 5, 6, 7, and 8, a SPECT imaging system similar to SPECT imaging system 34 locates and images ROI 38 by operating in two different configurations, and a secondary imaging system is not used.

Secondary imaging system 22 is assumed hereinbelow, by way of example, and unless otherwise stated, to comprise an X-ray CT machine.

CT machine 22 has an operational volume 23 which is typically substantially fixed with respect to the machine. If an object is placed within its operational volume, machine 22 is able to form images of the object. To determine ROI 38, patient 26 is placed in operational volume 23, and, as described in more detail below, the images generated by the CT machine are used to locate the ROI.

CT machine 22 is operated by an imaging facility processor 28 under overall control of an operator 32 of the facility. Processor 28 uses a memory 29 wherein is written, inter alia, operating software 31 for performing imaging, as described hereinbelow. Software 31 may be provided to facility 20 as a computer software product in a tangible form on a computer-readable medium such as a CD-ROM, or as an electronic data transmission, or as a mixture of both forms. In some embodiments of the present invention, memory 29 comprises a correspondence table 27, described below.

Typically, processor 28 is coupled to a graphic user interface 30 which allows operator 32 to see results of the operations performed by facility 20, as well as to issue commands to processor 28.

Facility 20 uses a movable bed 24 upon which patient 26 lays, according to instructions given to the patient by operator 32. Movable bed 24 is configured to be able to position ROI 38 of the patient so that CT machine 22 is able to generate images of the ROI.

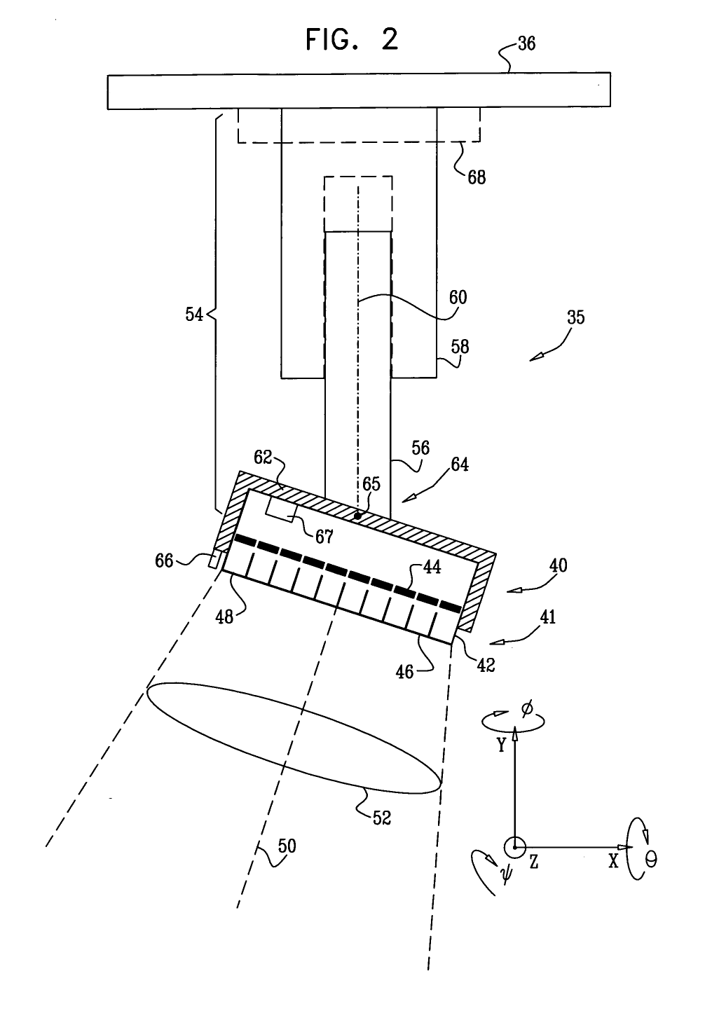

Primary imaging system 34 comprises a multiplicity of generally similar single photon counting imaging units 35 mounted on a bracket 36, and the system has an operational volume 39. Units 35 receive photons from concentrations of radio-isotopes in ROI 38. In one embodiment units 35 are fixedly mounted on bracket 36. Alternatively, units 35 are movably mounted on bracket 36. Further alternatively, primary imaging system 34 comprises a mixture of fixed and movably mounted units 35. Two units 35 are shown, by way of example, in FIG. 1. In some embodiments there are typically between approximately 3 and approximately 10 units 35 in facility 20. Units 35 are described in detail with respect to FIG. 2.

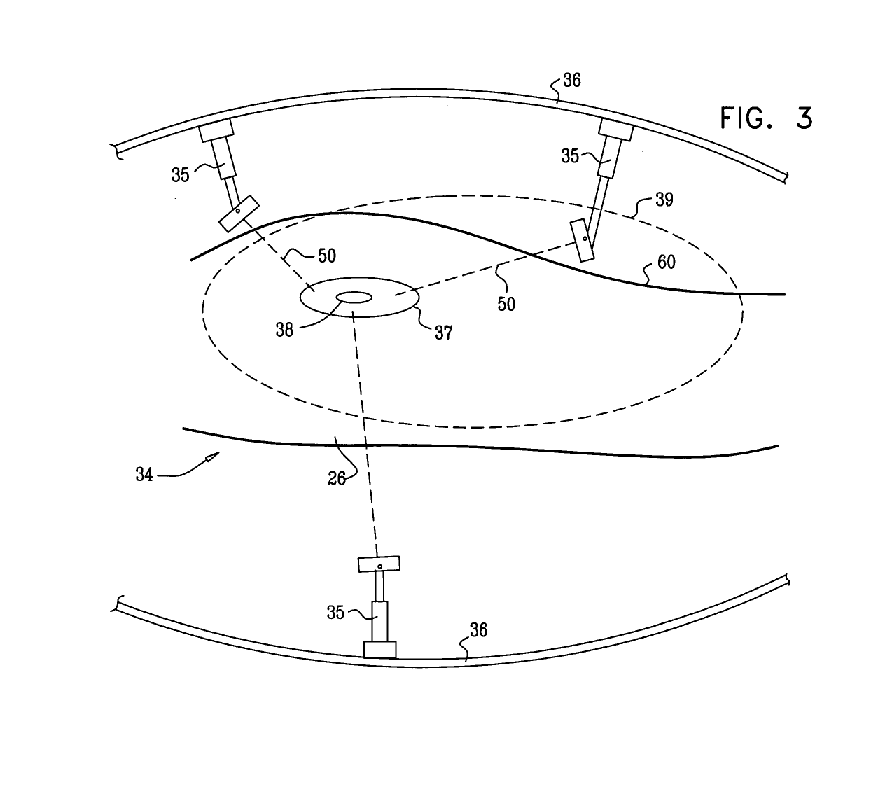

Primary imaging system 34 may be configured to operate in a mobile mode, typically a rotational mode, wherein stationary units 35 acquire signals after moving between well-defined positions, such as along bracket 36. Alternatively, primary imaging system 34 may be configured to operate in a static mode, wherein stationary units 35 acquire signals in one position only. Primary imaging system 34 is described in more detail with respect to FIG. 3.

FIG. 2 is a schematic diagram of a specific photon imaging unit 35, according to an embodiment of the present invention. Unit 35 comprises a single photon counting detector module 40, formed from a collimating system 41 in front of a two-dimensional array of photon counting detector elements 44, herein also referred to as detectors 44. Collimating system 41 comprises a collimator plate 42 having a plurality of collimator channels 46. A respective collimator channel 46 aligns with each detector element 44. In one embodiment detectors 44 comprise a square 4 cm×4 cm array of 256 elements, and collimator plate 42 has 256 collimator channels 46. Elements 44 typically comprise electrodes coupled to a semiconducting material such as Cadmium Zinc Telluride. Such detector elements are known in the art, and an example of a detector module having such detector elements is described in U.S. Pat. No. 5,847,398 to Shahar, et al., which is incorporated herein by reference. Alternatively, detector elements 44 may be formed from scintillators. While elements 44 may detect gamma rays and/or X-rays, herein, unless otherwise stated, elements 44 are assumed to be configured to detect gamma rays.

Collimator channels 46 all have substantially the same shape and size, and are herein by way of example assumed to be right prisms having a rectangular base. Thus, collimator plate 42 defines a front plane surface 48, herein also termed a module bounding surface 48, of module 40. Module 40 has an axis of symmetry 50 normal to surface 48, and the alignment of collimator channels 46 with elements 44 causes module 40 to have a solid angle of acceptance 52 for photons, the solid angle also having axis 50 as an axis of symmetry. Angle 52 is also referred to herein as the viewing angle of the module. Gamma ray emitters within solid angle 52 are thus detected by module 40, whereas if the emitters are outside the solid angle they are not detected by the module.

Module 40 is coupled to an adjustable module mount 54. Mount 54 comprises a cylindrical extensible arm 56, which slides within a cylindrical holder 58, and which also rotates around a common axis 60 of the holder and the arm. A rotatable plate 62 is coupled to an end 64 of arm 56, the plate having an axis of rotation 65 which is at right angles to axis 60, and module 40 is fixedly connected to the plate. Actuators for mount 54, which effect the extension and rotation of arm 56 and the rotation of plate 62, are under overall control of processor 28. By controlling the actuators of mount 54, processor 28 is also aware of the coordinates of the location and the orientation of module 40. For clarity, the actuators and their cabling, as well as other cabling used for operating unit 35, are not shown in FIG. 2. Module 40 thus has one degree of linear freedom and two degrees of rotational freedom, the latter allowing processor 28 to align axis 50 of the module in substantially any direction.

In some embodiments of the present invention, a position detector 66 is fixed to plate 62, and is arranged to be able to detect the presence of an object in front of surface 48 by generating a signal in response to the object's presence. Detector 66 is operated by processor 28, and the processor is able to analyze the signal from the detector in order to measure the distance of the object from surface 48. Detector 66 may operate by contact with a surface of the object, or in a non-contact mode of operation. Both types of detectors are well known in the art: for example, a contact detector may comprise a microswitch, a non-contact detector may operate by measuring capacitance between the detector and the object.

Detector 66 provides information to the processor about the coordinates of surface 48. This information, together with the information about the ROI derived from the secondary system, is used by the processor to align surface 48 toward the ROI while maintaining the distance between surface 48 and the object to be as small as possible. In operating system 34, the distance of each unit 35 is controlled to follow the contours of the object being imaged. It should be understood that such control may be applied whether the system acquires the image using a mobile, typically a rotational, scanning mode or using a static scanning mode.

In alternative embodiments of the present invention, the function of detector 66 is implemented by existing elements of unit 35, so that there is not a separate physical detector. For example, the presence of the object in front of surface 48 may be detected by measuring the capacitance between collimator 42 and the object. Processor 28 is configured so that it uses signals from detector 66, or equivalent signals if detector 66 is not implemented in unit 35, to position surface 48 as close as possible to the object surface, to follow the contours of the object surface as the unit operates, and to align the viewing angle of the unit with the ROI. Typically processor 28 is configured to position surface 48 a pre-set distance, typically in a range between of the order of 1 cm and 0 cm from the object surface.

Unit 35 is fixedly mounted to bracket 36. Alternatively, unit 35 is movably mounted by one or more actuators 68, indicated by broken lines, to bracket 36. Depending on which actuators 68 are used, the movable mounting may apply further rotational and/or linear displacements to unit 35, so that unit 35 may have a total of three translational and three rotational degrees of freedom. The six degrees of freedom are illustrated in FIG. 2 as translations along the mutually orthogonal x, y, z axes, and rotations θ, φ, Ψ about the respective axes.

The ROI behaves generally as an assembly of point sources, so that the photon flux at a given detector, generated by the concentrations of radio-isotopes in the ROI, decreases as the square of the distance of the detector from the ROI. The photon flux at the detector is further reduced by the collimation of photons traversing the collimator channel in front of the detector. In order that the photon flux received at the detector is sufficient, in other words in order that the signal to noise ratio (SNR) at the detector is large enough, it is advantageous to position detector modules as close to the ROI as possible. The size of the detector module used in unit 35 enables all the detectors in the modules to be simultaneously positioned close to the ROI. Furthermore, by attaching the detector module to an adjustable mount, all detectors in the module can be positioned to receive an optimal photon flux simultaneously from the ROI, and thus simultaneously achieve an optimal SNR.

In some embodiments of the present invention, imaging unit 35 comprises a processing module 67. Processing module 67 may be configured to operate unit 35, typically under overall control of processor 28, so as to reduce the computing power needed by processor 28.

FIG. 3 is a schematic diagram showing primary imaging system 34 in relation to patient 26 and ROI 38, according to an embodiment of the present invention. By way of example, three units 35 of the system are shown. Primary imaging system 34 comprises overall limiting operational volume 39, which is generally similar in properties to operational volume 23, so that imaging system 34 is able to form images of objects placed within volume 39. In addition to overall limiting operational volume 39, system 34 comprises an adjustable operational volume 37, which is an adjustable region included in overall volume 39. Adjustable volume 37 comprises a region where the axes of symmetry 50 and the solid angles of acceptance 52 of units 35 meet and/or overlap. Thus, the location and size of adjustable operational volume 37 may be adjusted, within the overall limiting volume 39, by setting the location and orientation of each module 40 of system 34.

Depending on which secondary imaging system 22 is used in facility 20 (FIG. 1), operational volume 23 and overall limiting volume 39 may or may not at least partly overlap. For example, if secondary imaging system 22 comprises an MRI CT machine, the two volumes may have to be physically separate, because of limitations inherent in the layout of the MRI machine. If secondary imaging system 22 comprises an X-ray CT machine, the two volumes may be generally the same.

Patient 26 is assumed to have an outer surface 60, which may typically comprise the skin of the patient and/or clothes, such as a hospital gown, that the patient is wearing. As is explained in more detail with respect to flowchart 80 below, processor 28 aligns and positions each unit 35 so that axis 50 of the unit is directed to ROI 38, and so that the module of the unit is as close as possible to ROI 38. In this case front surface 48 of each module 40 is close to, but typically does not contact, surface 60.

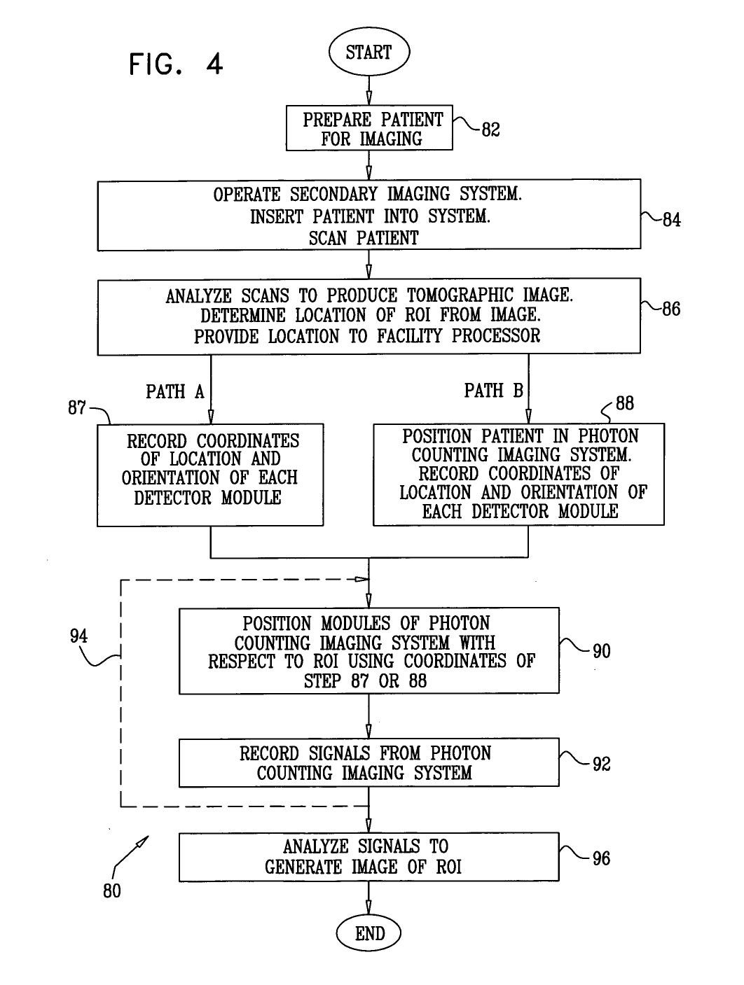

FIG. 4 is a flowchart 80 of a process used by processor 28, according to an embodiment of the present invention. In an initial step 82, operator 32 prepares patient 26 for imaging by administering a radio-isotope to the patient. The radio-isotope is typically in the form of a radio-pharmaceutical specific to the region of interest to be imaged. Examples of radio-isotopes which may be used are described in the Background of the Invention. During the remainder of the steps of flowchart 80, patient 26 is substantially immobile on bed 24.

In a first imaging step 84, during which the secondary imaging system operates, operator 32 inserts patient 26 into machine 22 by moving bed 24. The insertion is performed so that a region of patient 26 that includes ROI 38, is in operational volume 23 of CT machine 22. The operator then activates machine 22, which takes multiple X-ray scans of the region.

In an analysis step 86, processor 28 processes the multiple X-ray scans to produce a corresponding X-ray tomographic image, and the processor automatically determines coordinates of a location of ROI 38 from the tomographic image. Alternatively, operator 32 determines the coordinates of the location of ROI 38 from the tomographic image and provides the coordinates of the ROI to processor 28. The processing of the scans, and the determination of the location of the ROI, typically initiates before all the X-ray scans have been performed.

If operational volume 23 of the secondary imaging system and operational volume 39 of the primary imaging system are generally the same, then flowchart 80 follows a path “A.” If the two volumes are different, then the flowchart follows a path “B.”

In path A, in a step 87, processor 28 records the coordinates of the location and orientation of each module 40, using their respective actuators. Path A then continues to step 90 below.

In path B, in a repositioning step 88, processor 28 moves patient 26 by moving bed 24 into overall limiting volume 39 of the primary SPECT system. Alternatively, operator 32 removes patient 26 from the CT machine, by moving bed 24, and positions the patient for imaging by the primary imaging system. The repositioning is performed in a controlled manner, by moving bed 24, so that processor 28 is aware of the new location of ROI 38. The repositioning ensures that ROI 38 is in overall limiting volume 39 of system 34. In addition, processor 28 records the coordinates of the location and orientation of each module 40, using their respective actuators. Path B then continues to step 90.

In a second imaging step 90, in which primary imaging system 34 operates, processor 28 positions each module 40 of the primary imaging system so that operational volume 37 encloses ROI 38. Thus, for each unit 35, the processor operates the actuators of mount 54 so that the module of the unit moves from the initial known location and orientation, recorded in step 87 or 88, to a final known location and orientation. In the final location and orientation axis 50 of the module is approximately aligned with ROI 38, the coordinates of which have been derived by the secondary imaging system, as described above in step 86. In addition, the processor operates the actuators so that surface 48 is at the pre-set distance for module 40 of the unit.

In a step 92, when a given unit 35 is in position, processor 28 records signals generated at the module of the unit by photon absorption. The recording of the signals may be for a time that has been set by operator 32. Alternatively, the recording of the signals may continue until measurements by the processor on the signals indicate that module 40 has received sufficient photons for the processor to be able to generate an acceptable image from the signals.

Optionally, for example if system 34 operates in a mobile mode, steps 90 and 92 may be repeated for one or more specific units 35. Broken line 94 indicates the repetition of the steps. The steps are repeated by repositioning a unit to a new position, as is described for step 90, and then recording signals from the unit as is described in step 92.

In an image production step 96, processor 28 analyzes the signals generated in step 92, and forms one or more SPECT images of ROI 38 from the signals. Methods for generating SPECT images are well known in the art. Typically, operator 32 views the images in interface 30.

Flowchart 80 then ends.

Embodiments of the present invention combine two imaging processes for quickly and accurately producing single photon counting images of a particular ROI. A first imaging process, exemplified in flowchart 80 by a CT imaging process, locates the ROI. A second imaging process positions small single photon counting modules with respect to the ROI. Once the modules have been correctly positioned, the processor receives signals from the modules and generates an image of the ROI from the signals.

The embodiments described above illustrate how the two imaging processes are performed by two separate imaging systems, so as to quickly generate a final single photon image of a desired region of interest. It will be understood that the time for generation of the final SPECT image is considerably reduced, compared to other SPECT systems that give a comparable quality image, since in embodiments of the present invention the CT imaging process is used in parallel for location of the ROI. The end result is that a CT image and a fine quality SPECT image are produced in an overall time that is significantly less than prior art systems which operate independently.

As is described in more detail below with respect to FIGS. 5, 6, 7, and 8, alternative embodiments of the present invention dispense with two separate imaging systems for the two imaging processes. Rather, the alternative embodiments comprise one or more photon imaging units, generally similar to units 35, implemented as one imaging system operable in two different configurations. A first configuration is used for the first imaging process to locate the ROI, and a second configuration is used for the second imaging process to image the ROI.

FIG. 5 is a schematic diagram of a photon imaging unit 135, according to an alternative embodiment of the present invention. Apart from the differences described below, the operation of unit 135 is generally similar to that of unit 35 (FIG. 2), such that elements indicated by the same reference numerals in both units 35 and 135 are generally identical in construction and in operation. Unit 135 comprises a single photon counting detector module 140, formed from an adjustable collimating system 141 in front of detectors 44. In contrast to collimating system 41, which generates for its unit 35 one specific solid angle of acceptance 52, collimating system 141 is adjustable and has different solid angles of acceptance. Examples of different types of adjustable collimating systems are described in U.S. patent application titled “Variable Collimation in Radiation Detection,” filed 28 Mar. 2007, which is assigned to the assignee of the present invention and which is incorporated herein by reference.

Collimating system 141 is herein, by way of example, assumed to be able to operate in two configurations: a first unit configuration 144 having one collimator plate 146 in front of detectors 44, and a second unit configuration 148 having plate 146 and a second collimator plate 150 in front of the detectors. Plates 146 and 150 are generally similar to plate 42, both plates having the same number of collimator channels 147 as the number of detector elements 44. Collimator channels 147 of plates 146 and 150 are assumed to have generally the same cross-section and layout as collimator channels 46. However, the height of the collimator channels for plate 146 may be different from the height of the channels for plate 150.

In first unit configuration 144 the one plate 146 is aligned with detectors 44 and provides the detectors with a relatively large solid angle of acceptance 152. In second unit configuration 148 plates 146 and 150 are aligned with each other and with detectors 44, and provide the detectors with a relatively narrow solid angle of acceptance 154. In the first unit configuration, plate 146 defines a first configuration module bounding surface 143. In the second unit configuration, plate 150 defines a second configuration module bounding surface 149.

In embodiments where position detector 66 is implemented, signals from the detector may be used to measure the distances of surfaces 143 and 149 from an object, substantially as explained above for unit 35. Alternatively, as is also explained above with reference to unit 35, existing elements of unit 135 may be used to measure the distances of surfaces 143 and 149 from the object.

Unit 135 comprises mount 54, described above with respect to unit 35. As explained above, processor 28 controls the actuators of the mount. Thus, the processor is aware of the coordinates of the location and orientation of module 140, as well as the coordinates of surfaces 143 and 149.

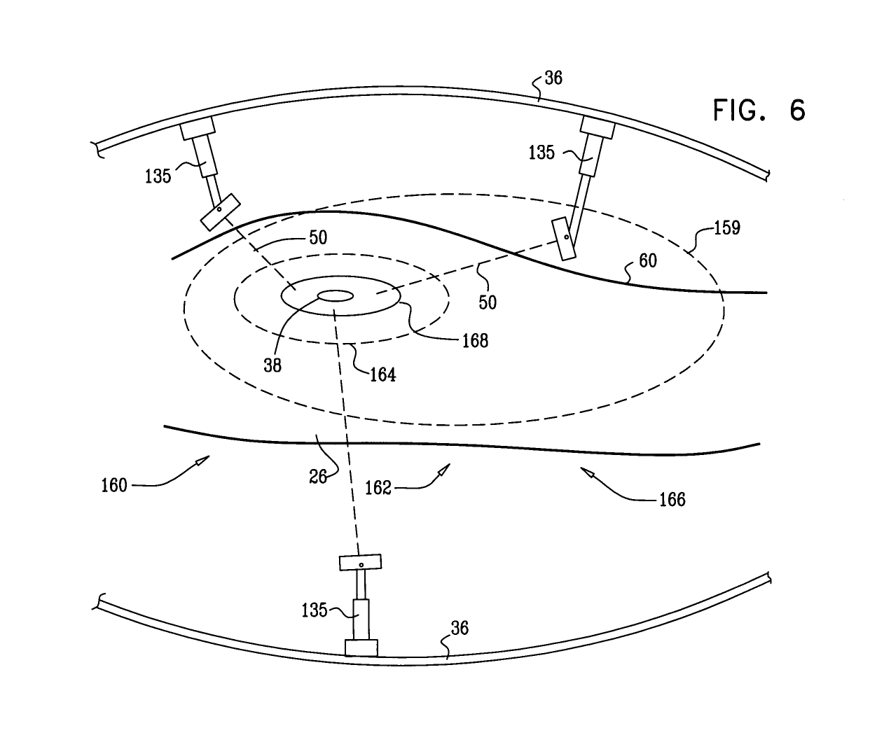

FIG. 6 is a schematic diagram illustrating a SPECT imaging system 160 in relation to patient 26 and ROI 38, according to an embodiment of the present invention. Apart from the differences described below, the operation of system 160 is generally similar to that of system 34 (FIG. 3), such that elements indicated by the same reference numerals in both systems 34 and 160 are generally identical in construction and in operation. In place of units 35, system 160 comprises units 135, described above with reference to FIG. 5. Typically, system 160 comprises more than three units 135, but for clarity only three are shown in FIG. 6.

Imaging system 160 has an overall limiting volume 159, which is generally similar in properties to overall limiting volume 39 (FIG. 3), so that imaging system 160 is able to form images of objects placed within volume 159. Imaging system 160 is arranged to be able to operate in two different system configurations. In a first system configuration 162 some of units 135, typically all or the majority of the units, are arranged to operate in first unit configuration 144. First system configuration 162 is also herein termed coarse configuration 162. In coarse configuration 162 system 160 has a first adjustable operational volume 164. In a second system configuration 166 some of units 135, typically all or the majority of the units, are arranged to operate in second unit configuration 148. Second system configuration 166 is also herein termed fine configuration 166. In fine configuration 166 system 160 has a second adjustable operational volume 168. Operational volumes 164 and 168 have generally similar properties to operational volume 37. However, operational volume 164 is larger than, and typically completely encloses, operational volume 168. Overall limiting volume 159 comprises all possible volumes 164 and 168.

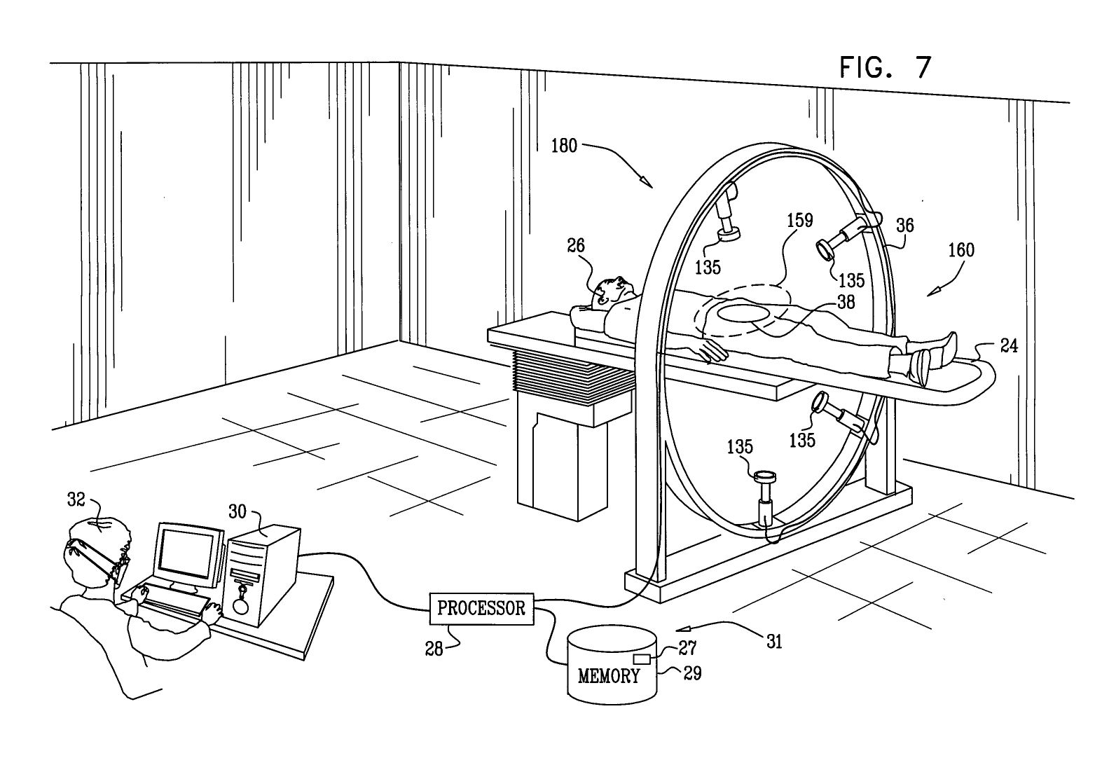

FIG. 7 is a schematic diagram of an imaging facility 180, according to an embodiment of the present invention. Apart from the differences described below, facility 180 is generally similar to facility 20 (FIG. 1), such that elements indicated by the same reference numerals in facility 20 and 180 are generally substantially similar. In facility 180 there is no secondary imaging system 22, and single photon counting imaging system 160 replaces primary imaging system 34. Single photon imaging system 160 is operated by processor 28 in its two configurations to locate and image ROI 38, as described below in reference to flowchart 200.

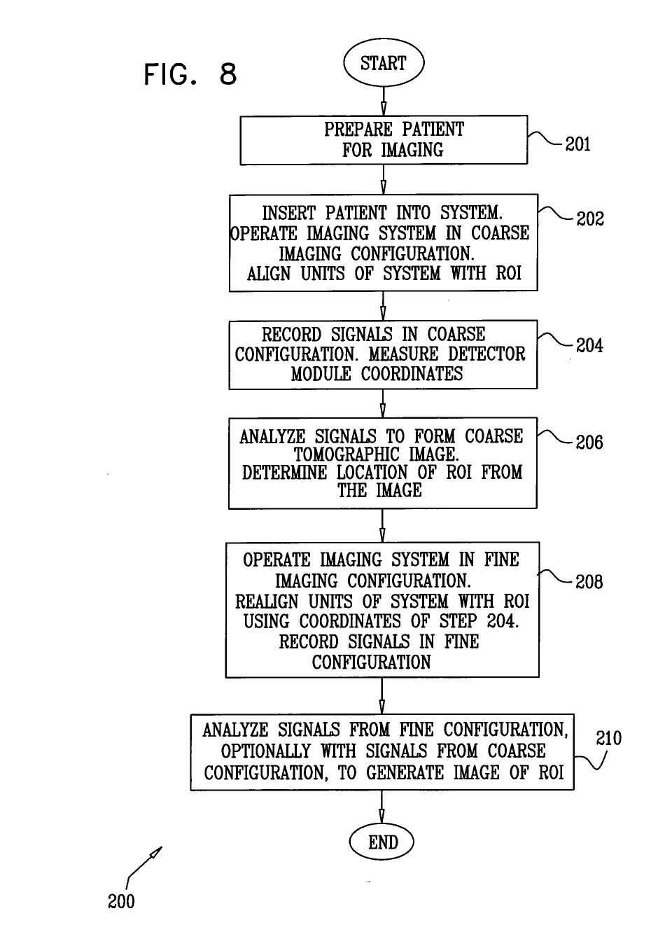

FIG. 8 is a flowchart 200 of a process used by processor 28 in facility 180, according to an alternative embodiment of the present invention. In flowchart 200, system 160 may operate in a mobile mode or in a static mode.

Step 210, in which patient 26 is prepared for imaging, is substantially as described above for step 82 of flowchart 80. During the remainder of the steps of flowchart 200, patient 26 is substantially immobile on bed 24.

In an alignment step 202, operator 32 inserts a region of patient 26 that includes ROI 38 into overall limiting volume 159 by moving bed 24. Processor 28 or the operator then activates system 160 into its coarse configuration 162. Processor 28 aligns units 135 so that first adjustable operational volume 164 includes the region with ROI 38.

In a first imaging step 204 processor 28 records signals generated at each module 40 of system 160 by photon absorption. The recording of the signals may be for a time that has been set by operator 32. Alternatively, the recording of the signals may continue until measurements by the processor on the signals indicate that modules 40 have received sufficient photons for the processor to be able to generate an acceptable image from the signals. Processor 28 also records the coordinates of the location and alignment of each module.

In an analysis step 206, processor 28 processes the signals to form a coarse tomographic image of the region including ROI 38. From the coarse image and the known coordinates of the detector modules, operator 32 and/or processor 28 determine a location of ROI 38.

In a second imaging step 208, operator 32 activates system 160 into its fine configuration 166. If required processor 28 realigns units 135 so that ROI 38 is within second adjustable operational volume 168, using the coordinates determined in step 204. Processor 28 again records signals generated at each module 40 of system 160 by photon absorption, substantially as described above for step 204.

In an image production step 210, processor 28 analyzes the signals generated in step 208, optionally together with the signals previously generated in step 204, and forms one or more SPECT images of ROI 38 from the signals. Typically, operator 32 views the images in interface 30.

Flowchart 200 then ends.

In the alternative embodiments using one photon counting imaging system operating in two configurations, substantially the same advantages of reduction in time required to generate the final image of the ROI apply, as for the case for the two imaging systems. The reduction of time for the alternative embodiments arises because in the first unit configuration the number of photons absorbed by unit 135 in a relatively short time period is sufficient to form an image from which the ROI can be located. In addition, the image information from the coarse and the fine images may be combined to further reduce the acquisition time for the fine image.

In all embodiments, the SPECT systems described above use a multiplicity of adjustable single photon counting detector modules which are relatively small. The size of the modules allows them to be individually positioned so that all their respective detector elements are each as close as possible to the surface of a patient, and are aligned with the ROI of the patient. This contrasts with SPECT systems using one large single photon counting detector module, which by its very size may at best only position a small portion of its detector element close to the surface of the patient and aligned with the ROI.

The embodiments above illustrate that one photon counting imaging system operating in two configurations improves the time taken to produce a final image of an ROI. The one photon imaging system may be arranged to operate in more than two configurations. For example, in SPECT imaging system 160 coarse configuration 162 may comprise all units 135 operating in first unit configuration 144, fine configuration 166 may comprise all unit 135 operating in second unit configuration, and there may be a third system configuration where some units 135 operate in the first unit configuration, and the other units 135 operate in the second unit configuration. Alternatively or additionally, some units 135 may be arranged to have collimating systems 141 that have more than two unit configurations, and different system configurations of system 160 may be arranged using the different unit configurations available. By using more than two system configurations, the operator/processor 28 of the imaging facility may further reduce the time taken to obtain a final image of the ROI.

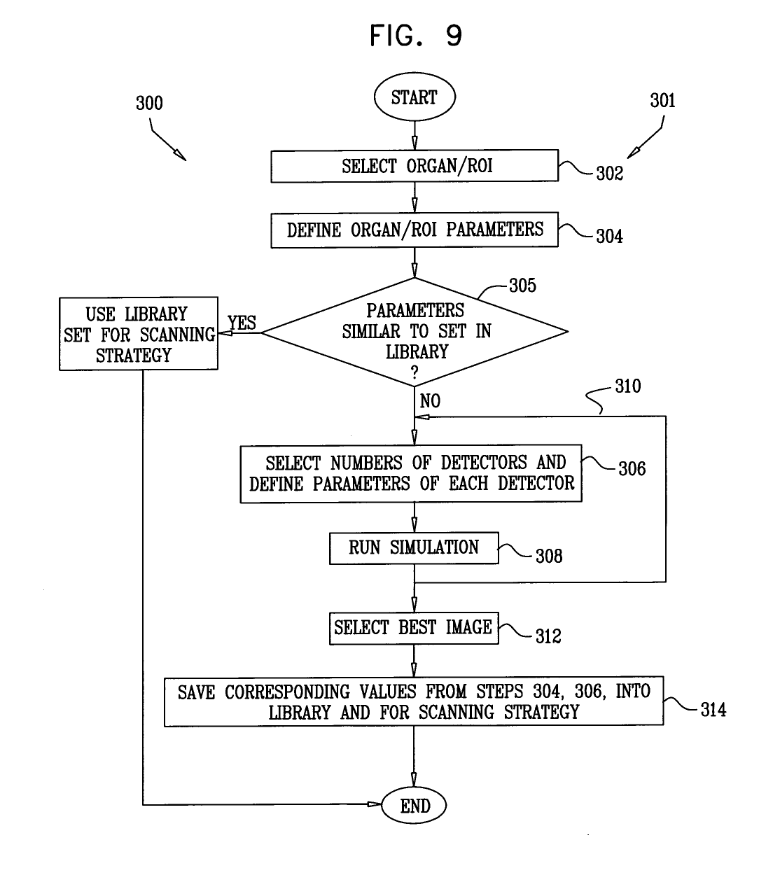

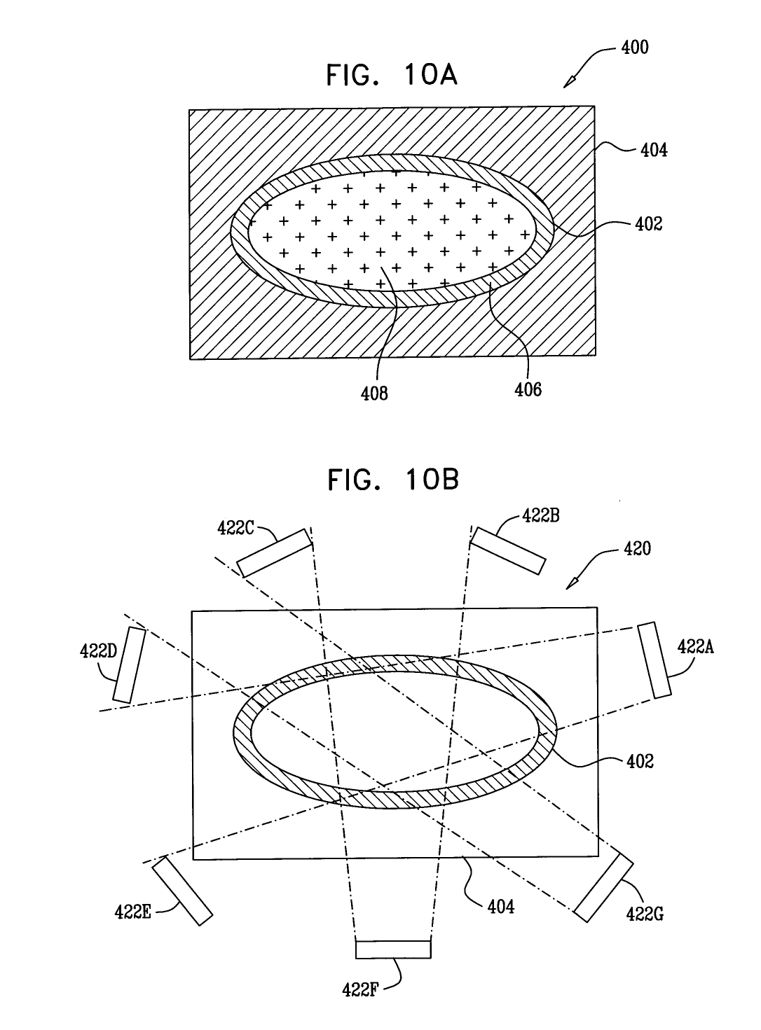

FIG. 9 is a flowchart 301 of a simulation process 300 that may be applied in facility 20 and/or facility 180, and FIGS. 10A and 10B show diagrams illustrating the process, according to embodiments of the present invention. In both facilities it is advantageous to reduce the time spent in imaging patient 26 to a minimum, without disadvantageously altering other imaging factors, such as increasing the concentration of radio-isotope administered to the patient. The results of simulation process 300 may be applied off-line, before patient 26 is imaged on one of the facilities, as well as on-line, during imaging of the patient, to provide such a reduction in imaging time. Both procedures are described below.

Process 300 comprises a computer simulation that may be performed by processor 28 (FIG. 1 and FIG. 7) under control of operator 32. The process simulates images produced by imaging system 34 or system 160, by investigating different possible scanning strategies for the systems. A scanning strategy comprises a definition, during scanning of patient 26, of a number and an overall topology of the detectors of units 35 and 135, how each of the detectors is oriented and/or translated from an initial position defined by the topology, and an acquisition time period during which each of the detectors is at the given orientation and position. During the acquisition time, signals generated by a detector in response to photons interacting with the detector, are recorded. An object of process 300 is to find an optimal scanning strategy for the detectors, so that imaging time is reduced as much as possible. Process 300 is typically performed on different simulated organs/ROIs, such as a simulated heart, liver, or other organ, or a simulated ROI such as a part of a limb.

While the description of process 300 herein is directed to SPECT systems, it will be appreciated that, mutatis mutandis, the simulation process may be applied to other scanning systems, typically systems having multiple detectors such as ultrasound scanning systems and/or the other modalities for locating an ROI referred to above.

In an initial step 302, operator 32 selects an organ or ROI to be simulated.

The remaining steps of flowchart 301 may be performed by operator 32, typically at least partly using processor 28. In some embodiments, at least some of the steps may be performed substantially automatically by processor 28, with little or no operator intervention. Steps where such automatic performance is possible, and/or where operator intervention is required, will be apparent to those having ordinary skill in the art.

In a parameter definition step 304, applicable parameters of the organ/ROI selected in step 302 are defined. Applicable parameters comprise:
Relevant external dimensions of the organ/ROI, and the position and orientation of the organ/ROI with respect to neighboring organs/ROIs. In some embodiments, the operator and/or processor 28 may choose a geometric figure as a first approximation of the organ/ROI, such as an ellipsoid for the heart. In another embodiment, the chosen approximation may be based on results of a prior examination of the organ/ROI of patient 26. For example, the results may be generated from a prior ultrasound examination that may include forming an ultrasound image of the organ/ROI.
Internal dimensions of elements of the organ/ROI, as well as radio-isotope take-up coefficients and radiation absorption coefficients of the elements.
Radiation absorption coefficients of elements surrounding the organ/ROI.
Other relevant parameters of the elements surrounding the organ/ROI, such as dimensions of the elements, and locations of the elements in relation to the organ/ROI.

In FIG. 10A a diagram 400 illustrates parameter definition step 304. An ellipsoid 402, simulating an organ such as the heart, is located within a generally box-like region 404. Ellipsoid 402 has an outer layer 406 simulating the outer wall of the heart and a region 408, within the outer layer, simulating chambers of the heart. As stated above, in step 304 operator 32 and/or processor 28 defines dimensions, orientations, take-up coefficients and absorption coefficients of the various parts of ellipsoid 402, layer 406, and region 404.

Returning to flowchart 301, in a comparison step 305, the parameters determined in step 304 are compared with a library of sets of organ/ROI parameters stored in memory 29. Generation of the library is described in more detail below. If the parameters determined in step 304 are similar, within predefined tolerances, to one of the sets of the library, then the flowchart continues to a scanning strategy step 307. If the parameters are not similar to any of the sets, the flowchart continues to step 306.

In a detector setup step 306, the numbers of detectors to be used in the simulation are selected. In addition, parameters for each detector, the numbers and parameters defining a scanning strategy, are chosen. Typically for each detector the parameters include a field of view of the detector, such as solid angle 52, solid angle 152, or solid angle 154, (FIGS. 2, 5) an initial location, and an initial direction of axis of symmetry 50. The parameters also include changes of position and/or of orientation of each detector that are to be implemented for the scanning strategy, an acquisition time during which each set of positions and orientations is to be implemented, as well as an order within the strategy in which each set is to be implemented.

In FIG. 10B a diagram 420 illustrates a detector setup as provided by step 306, and as applied to ellipsoid 402. By way of example, 7 simulated detectors 422A, 422B, . . . , 422G are assumed to be selected. In diagram 420 three exemplary fields of view are illustrated for detectors 422A, 422F, and 422G. It will be understood that in step 306 fields of view for all simulated detectors, as well as the other parameters for each detector described above, are provided.

In a run simulation step 308, processor 28 simulates an image generated for the system set up in steps 302, 304, and 306. The simulation is performed on a statistical basis, assuming a half-life of the radio-isotope being used, concentrations of radio-isotope, take-up coefficients of elements in the simulated organ/ROI, and absorption factors for elements surrounding the organ/ROI. Using these factors and collimator properties of each detector, the processor is able to simulate if a given dis-integration of a radio-isotope nucleus generates a signal at a specific detector. From the simulated signals, the processor generates an image of the organ/ROI that changes over time as the radio-isotope continues to disintegrate. The simulation is performed assuming acquisition times for the detectors for each set of positions and orientations defined in step 306, and completes when the overall time defined in step 306 has been reached.

As indicated by line 310, steps 306 and 308 are typically implemented repeatedly, each repetition changing one or more of the parameters applied in step 306.

In a select step 312, operator 32 selects which of the images produced in simulation step 308 is closest to the expected image. In the example described above, the expected image corresponds to diagram 400. Alternatively or additionally, processor 28 may be programmed to compare the images produced with the expected image using methods known in the art, such as using a peak signal to noise ratio (PSNR) function, to find one or more images which are close to the expected image.

In a final, optimal strategy step 314, the parameters used in steps 304 and 306 to produce the image selected in step 312 are saved. The saved parameters are used to define an optimal scanning strategy for imaging the organ/ROI having parameters given by step 304. The optimal scanning strategy comprises:
The dose and type of radio-pharmaceutical required to implement the strategy.
The number of detectors;
The initial position of bracket 36 with respect to the patient, and the initial position of each detector on bracket 36;
Changes in x, y, z, θ, φ, Ψ values of the detectors;
The acquisition time for each detector at each group of x, y, z, θ, φ, Ψ values. Typically the acquisition times for all detectors for a given set of x, y, z, θ, φ, Ψ values are the same;
An order in which the groups are to be implemented;

Each group of x, y, z, θ, φ, Ψ values and its associated acquisition time is herein also referred to as a scan, and a scanning strategy comprises an assemblage of scans. Typically the scans of a given optimal scanning strategy are ordered so that the time to proceed from one scan to a following scan in the strategy is minimized. Such minimization may be accomplished by ensuring that in proceeding from one scan to the next, detector motion is substantially uni-directional.

The assemblage of scans for the optimal scanning strategy, together with a correspondence between the assemblage of scans and the corresponding organ/ROI parameters of step 304, is saved in memory 29. As flowchart 301 continues to be implemented for different organs/ROIs, a table 27 of such correspondences, i.e. a library of correspondences between sets of organ/ROI and their respective assemblage of scans, is stored in memory 29. The library is used in comparison step 305.

Scanning strategy step 307 is performed if the comparison in step 305 shows that an applicable set of parameters, saved in step 314 in an earlier implementation of the flowchart, exits. In scanning strategy step 307 the applicable set of parameters is used for the scanning strategy of the organ/ROI.

Performing simulations of scanning strategies according to flowchart 300 allows operator 32 to develop optimal scanning strategies for different organs and ROIs. In addition, differences in parameters for each of the organs or ROIs may be simulated, and corresponding optimal scanning strategies developed. For example, the human liver varies significantly in dimensions from person to person, so that flowchart 300 may be applied to find optimal scanning strategies for the differently dimensioned livers.

The simulations also allow different topologies for detectors to be investigated. For example, as illustrated in diagram 420, the inventors have found that good images are generated even if detectors 424A, 424B, . . . 422G are located so that respective fields of view for each detector, corresponding to solid angle 52 for a unit 35, substantially exclude any other detector. In such a topology, herein referred to as a “free-field-of-view” topology, detectors are substantially absent from the field of view of other detectors. Simulations have shown that the free-field-of-view topology provides good images of scanned regions without blind spots. However, it will be appreciated that the free-field-of-view type of topology is but one particular type of topology for the locations of simulated detectors, and that simulations as described herein may generate other topologies. Such other topologies are typically also substantially free from blind spots.

The simulations enable operator 32 to generate, verify, and refine models for producing the expected images of different organs/ROIs. From the models, image reconstruction may be performed to generate further images both by interpolation and extrapolation, as well as by fusion of several reconstructed images. Such model based reconstructed images may be used for comparison purposes, as is exemplified in a flowchart 800 described below.

It will be appreciated that as flowchart 301 continues to be used, the number of optimal scanning strategies in the library referred to in steps 305 and 314 increases. The increase in number of optimal scanning strategies means that the path through the flowchart following step 307 will be increasingly used, with a consequent saving in time for new patients, as well as increased throughputs for facility 20 and facility 180.

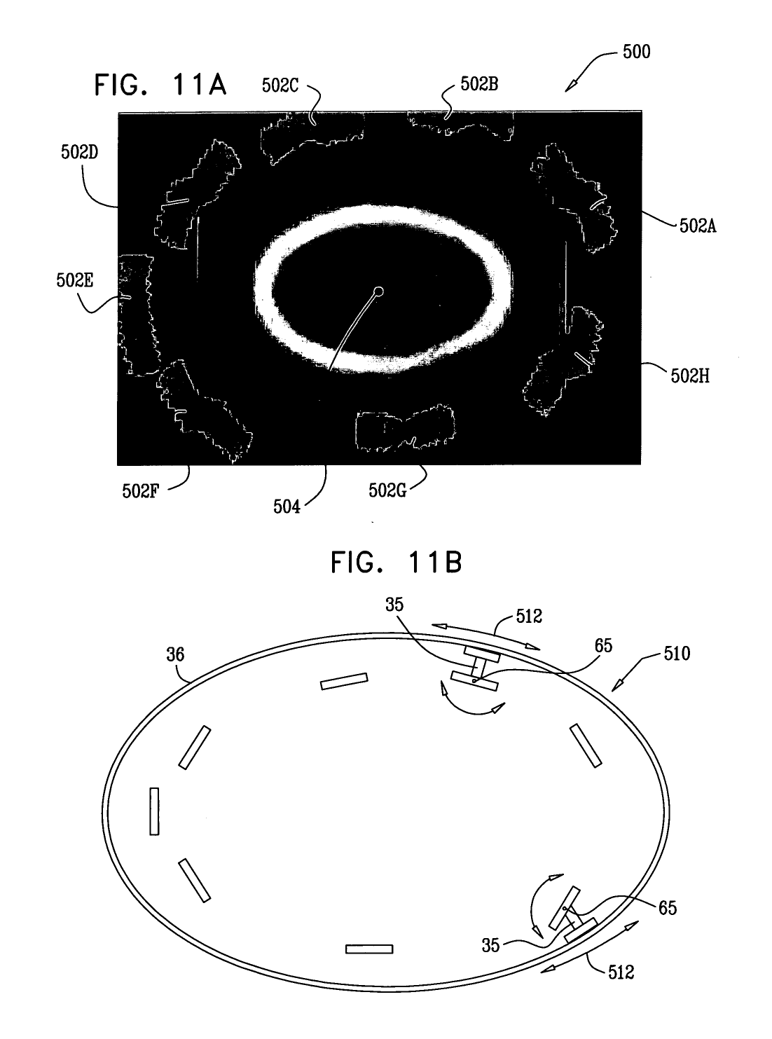

FIGS. 11A and 11B illustrate a simulated image, and a schematic corresponding setup in system 34, according to an embodiment of the present invention. A diagram 500 shows the simulated image of ellipsoid 402 (FIG. 10A). The image is generated by positioning 8 simulated detectors, 502A, 502B, . . . 502H around a point 504, corresponding to the center of the ellipsoid. The detectors are arranged in a free-field-of-view topology. In the simulation, each detector was rotated about its own axis, into 15 different positions, in 2 degree steps, and the rotations were performed without lateral movement of the detectors. All detectors were then moved approximately laterally, by rotating 2 degrees about point 504, and the 15 rotations of the detectors about their axes were repeated. The approximate lateral movements were repeated for five different lateral positions of each detector. The parameters for the detectors, as well as initial positions and orientations thereof, are incorporated into a scanning strategy for ellipsoid 402.

A diagram 510 illustrates schematically how units 35 in system 34 (FIG. 1) are arranged on bracket 36 to implement the simulated scanning strategy illustrated in diagram 500. For clarity, details of only two units 35 are shown in diagram 510, and detectors of the other units are illustrated as rectangles. As illustrated, each detector rotates about its own axis 65, corresponding to the 15 rotations of 2 degrees described above. In addition, as shown by lines 512, each unit 35 translates on bracket 36, corresponding to the five approximate lateral movements described above.

The simulation illustrated in diagram 500, and its implementation of diagram 510, use 8 detectors. However, a detector may be moved to different positions on bracket 36. Thus, if each detector moves to two positions on bracket 36, only four detectors may be needed to perform the required scanning strategy.

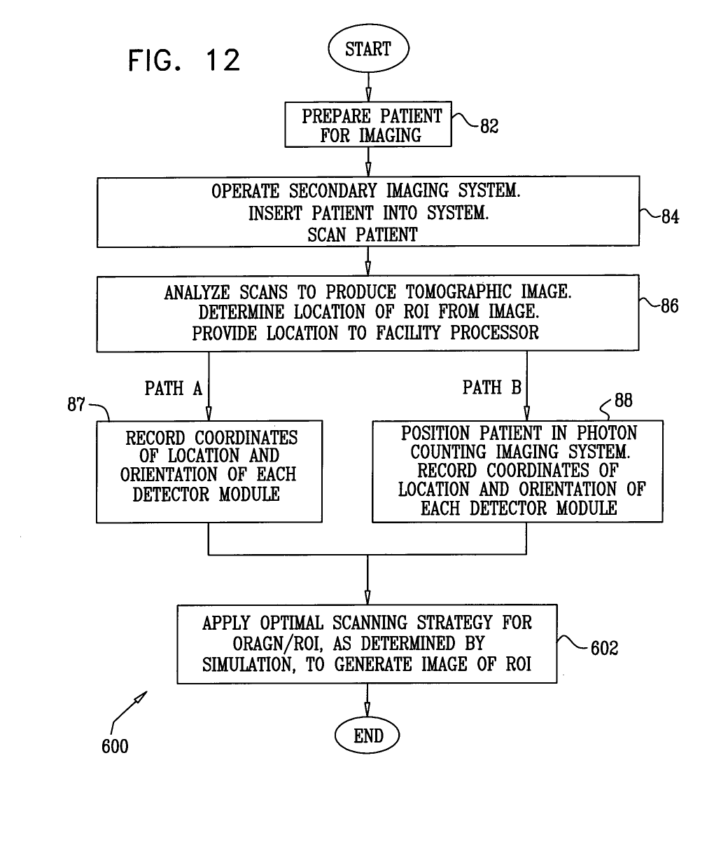

FIG. 12 is a flowchart 600 of a process used by processor 28, according to an embodiment of the present invention. Apart from the differences described below, flowchart 600 is generally similar flowchart 80 (FIG. 4), and steps indicated by the same reference numerals in both flowcharts are generally implemented in a substantially similar manner.

In the process described by flowchart 600, a secondary imaging system is used to determine the location of the ROI using steps 82 84, 86 and step 87 or step 88.

In a scanning step 602, which replaces steps 90, 92, and 96 of flowchart 80, an optimal scanning strategy for SPECT system 34 is chosen according to the identified ROI, and the scanning strategy is applied to the ROI. The ROI typically comprises an organ. The optimal scanning strategy has been determined from simulations described above with reference to FIGS. 9, 10, and 11.

In some embodiments, in step 602 an initial scan of the optimal scanning strategy is used to check that the alignment of the ROI is correct, by comparing results of the initial scan with results expected from the corresponding simulation. Depending on the comparison, elements of system 34 may be realigned by operator 32.

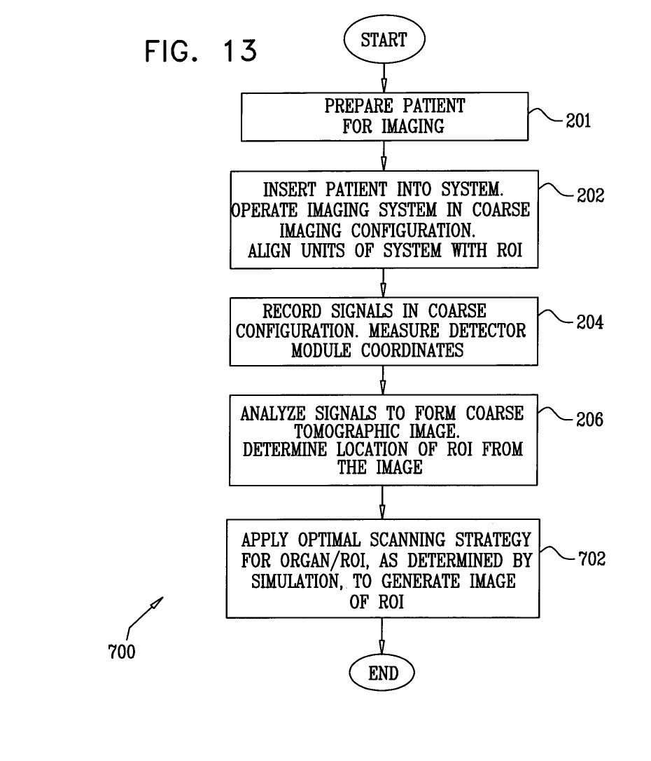

FIG. 13 is a flowchart 700 of a process used by processor 28, according to an embodiment of the present invention. Apart from the differences described below, flowchart 700 is generally similar to flowchart 200 (FIG. 8), and steps indicated by the same reference numerals in both flowcharts are generally implemented in a substantially similar manner.

In the process described by flowchart 700, only the SPECT imaging system is used. In steps 201, 202, 204, and 206 the system determines a location of an ROI, by operating the system in a coarse configuration. In a scanning step 702, which replaces steps 208 and 210 of flowchart 200, an optimal scanning strategy, determined by simulations described above, is implemented according to the ROI. Scanning step 702 is generally similar to step 602 (FIG. 12), so that in some embodiments an initial scan of the strategy is used to check and, if necessary, adjust the alignment of the ROI.

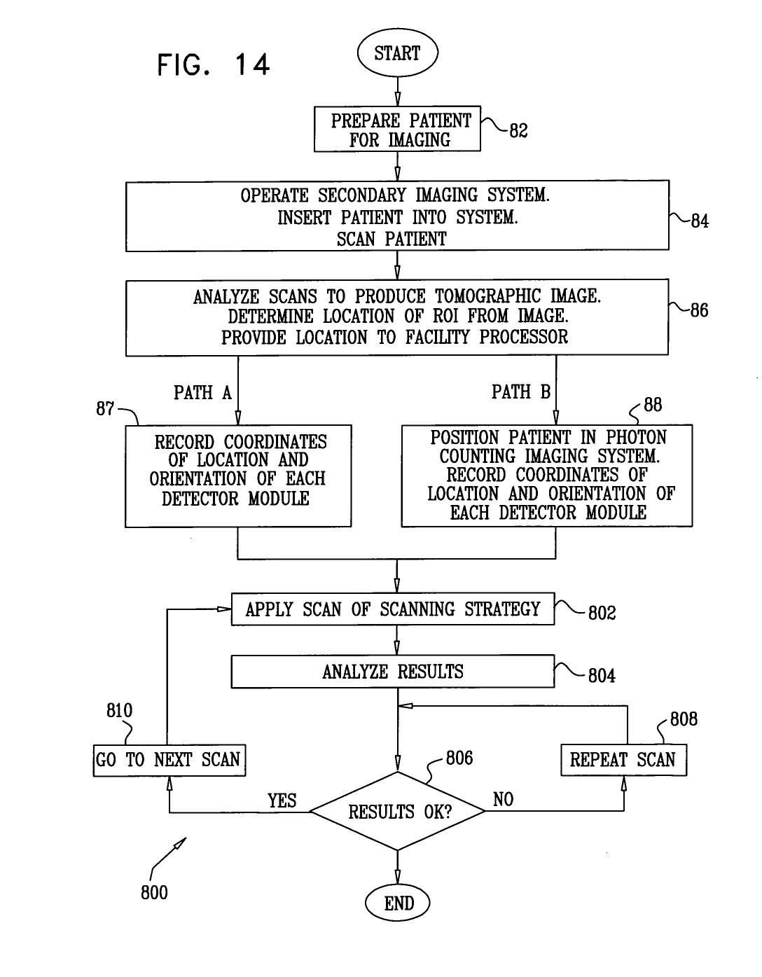

FIG. 14 is a flowchart 800 of a process used by processor 28, according to an embodiment of the present invention. Apart from the differences described below, flowchart 800 is generally similar flowcharts 80 (FIG. 4) and 600 (FIG. 12), and steps indicated by the same reference numerals in both flowcharts are generally implemented in a substantially similar manner.

As for flowchart 600, in the process described by flowchart 800, a secondary imaging system is used to determine the location of the ROI using steps 82 84, 86 and step 87 or step 88.

Once the location of the ROI has been determined, an optimal scanning strategy is selected, generally as described above for step 602. However, instead of applying the selected strategy as described in step 602, in a scan step 802 one scan, initially the first scan, of the strategy is performed.

The results of the scan are analyzed in an analysis step 804. The analysis comprises comparing the results obtained from signals generated by the detectors during the assigned acquisition time of the scan with expected results, as determined by the simulation used to determine the optimal scanning strategy, and/or as determined from model based reconstructed images referred to above.

In a comparison step 806, the analysis is used to determine if the scan results are acceptable. If they are, then in a subsequent scan step 810, parameters for the next scan of the strategy are implemented, and the flowchart returns to step 802.

If the scan results are not acceptable, then in a repetition step 808, the scan is repeated.

Flowchart 800 continues until all the scans in the strategy have been applied in step 802.

The inventors have found that embodiments of the present invention give good images, in short times, for ROIs comprising relatively static objects, as well as for ROIs comprising objects in motion. In the latter case, a good quality gated image, for instance for a beating heart, may be produced in a period of approximately 30 ms. The gating for the gated image may be generated by any convenient periodic signal known in the art. For the heart such signals include, but are not limited to, an ECG signal, the signals generated by the audible sound from the beating heart, typically using by a microphone, and a signal generated from an ultrasonic image of the beating heart.

In embodiments of the present invention processor 28 may comprise a single central processing unit, a distributed set of processing units, or a combination of the central unit and a distributed set. In some embodiments of the present invention processor 28 acts as a synchronizing computer, transmitting synchronizing signals to processing modules 67 in units 35 or 135, so that processor 28 and modules 67 operate in a “master-slaves” context.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

We claim:

1. A method for imaging a body, comprising:
scanning the body so as to generate an image thereof;
analyzing the image to determine a location of a region of interest (ROI) within the body;
providing a plurality of single photon counting detector modules, each of the single photon counting detector modules being configured to receive photons from a respective direction and to generate a signal in response thereto;
coupling each of the single photon counting detector modules to a respective adjustable mount;
adjusting each of the adjustable mounts so that the direction of the single photon counting detector module coupled thereto is aligned with respect to the location so as to receive radiation from the ROI;
operating each of the single photon counting detector modules to receive the photons from the ROI; and
in response to the signal generated by each of the single photon counting detector modules, generating a single photon counting image of the ROI.

2. The method according to claim 1, wherein scanning the body comprises scanning the body with an imaging system other than the plurality of single photon counting detector modules.

3. The method according to claim 2, wherein the imaging system comprises a computerized tomography imaging system, and wherein the image comprises a tomographic image.

4. The method according to claim 1, wherein each of the adjustable mounts is individually adjustable, and wherein adjusting the adjustable mounts comprises adjusting the mounts independently of each other.

5. The method according to claim 1, wherein adjusting each of the adjustable mounts comprises adjusting a distance of at least one of the modules from a surface of the body to be within a preset range.

6. The method according to claim 5, wherein the preset range is between of the order of 1 cm and 0 cm.

7. The method according to claim 1, wherein adjusting each of the adjustable mounts comprises measuring a location coordinate of at least one of the modules.

8. The method according to claim 1, wherein adjusting each of the adjustable mounts comprises measuring an orientation of at least one of the modules.

9. The method according to claim 1, wherein the plurality of the single photon counting detector modules are configurable in a multiplicity of system configurations wherein the modules receive the radiation from a multiplicity of respective different volumes enclosing the ROI.

10. The method according to claim 9, wherein scanning the body comprises arranging the plurality of the single photon counting detector modules in a first of the multiplicity to have a first volume enclosing the ROI, and wherein adjusting each of the adjustable mounts comprises arranging the plurality of the single photon counting detector modules in a second of the multiplicity to have a second volume enclosing the ROI and smaller than the first volume.

11. The method according to claim 1, wherein at least one of the single photon counting detector modules is operative in a first unit configuration wherein the at least one module is arranged to receive radiation from a first solid angle, and is operative in a second unit configuration wherein the at least one module is arranged to receive radiation from a second solid angle different from the first solid angle.

12. The method according to claim 1, wherein operating each of the single photon counting detector modules comprises operating the single photon counting detector modules in an operating mode selected from a group of modes comprising a rotational mode and a static mode.

13. The method according to claim 1, wherein the single photon counting image of the ROI comprises a single photon emission computerized tomography (SPECT) image.

14. Apparatus for imaging a body, comprising:
a plurality of single photon counting detector modules, each of the single photon counting detector modules being configured to receive photons from a respective direction and to generate a signal in response thereto;
a plurality of adjustable mounts respectively coupled to the single photon counting detector modules; and
a processor which is configured to analyze an image so as to determine a location of a region of interest (ROI) within the body, to adjust each of the adjustable mounts so that the direction of the single photon counting detector module coupled thereto is aligned with respect to the location so as to receive radiation from the ROI, to operate each of the single photon counting detector modules to receive the photons from the ROI, and in response to the signal generated by each of the single photon counting detector modules, to generate a single photon counting image of the ROI.

15. The apparatus according to claim 14, and comprising an imaging system, other than the plurality of single photon counting detector modules, which is configured to generate the tomographic image.

16. The apparatus according to claim 15, wherein the imaging system comprises a computerized tomography imaging system, and wherein the image comprises a tomographic image.

17. The apparatus according to claim 14, wherein each of the adjustable mounts is individually adjustable, and wherein adjusting the adjustable mounts comprises adjusting the mounts independently of each other.

18. The apparatus according to claim 14, wherein adjusting each of the adjustable mounts comprises adjusting a distance of at least one of the modules from a surface of the body to be within a preset range.

19. The apparatus according to claim 18, wherein the preset range is between of the order of 1 cm and 0 cm.

20. The apparatus according to claim 14, wherein adjusting each of the adjustable mounts comprises measuring a location coordinate of at least one of the modules.

21. The apparatus according to claim 14, wherein adjusting each of the adjustable mounts comprises measuring an orientation of at least one of the modules.

22. The apparatus according to claim 14, wherein the plurality of the single photon counting detector modules are configurable in a multiplicity of system configurations wherein the modules receive the radiation from a multiplicity of respective different volumes enclosing the ROI.

23. The apparatus according to claim 22, wherein analyzing the image comprises arranging the plurality of the single photon counting detector modules in a first of the multiplicity to have a first volume enclosing the ROI, and wherein adjusting each of the adjustable mounts comprises arranging the plurality of the single photon counting detector modules in a second of the multiplicity to have a second volume enclosing the ROI and smaller than the first volume.

24. The apparatus according to claim 14, wherein at least one of the single photon counting detector modules is operative in a first unit configuration wherein the at least one module is arranged to receive radiation from a first solid angle, and is operative in a second unit configuration wherein the at least one module is arranged to receive radiation from a second solid angle different from the first solid angle.

25. The apparatus according to claim 14, wherein operating each of the single photon counting detector modules comprises operating the single photon counting detector modules in an operating mode selected from a group of modes comprising a rotational mode and a static mode.

26. The apparatus according to claim 14, wherein the single photon counting image of the ROI comprises a single photon emission computerized tomography (SPECT) image.

27. Apparatus for imaging a region of interest (ROI) within a body having an outer surface, comprising:
a single photon counting detector module comprising:
a two-dimensional array of photon counting detectors, each of the detectors being configured to generate a signal indicative of a radio-isotope concentration in the ROI in response to a respective flux of photons received from the radio-isotope concentration; and
a plurality of collimator channels respectively coupled and aligned with the photon counting detectors in the two-dimensional array so that each of the photon counting detectors is able to receive the respective flux of the photons via its coupled collimator channel, the plurality of collimator channels being connected together so as to form a module outer surface; and
an adjustable mount to which the module is fixedly connected and which is configured to set an orientation of the module with respect to the ROI and to set a location of the module outer surface with respect to the outer surface of the body so that all of the photon counting detectors are able to simultaneously receive from the ROI the respective flux of the photons.

28. A method for imaging a region of interest (ROI) within a body having an outer surface, comprising:
providing a single photon counting detector module comprising a two-dimensional array of photon counting detectors, each of the detectors being configured to generate a signal indicative of a radio-isotope concentration in the ROI in response to a respective flux of photons received from the radio-isotope concentration;
coupling and aligning a plurality of collimator channels respectively with the photon counting detectors in the two-dimensional array so that each of the photon counting detectors is able to receive the respective flux of the photons via its coupled collimator channel;
connecting the plurality of collimator channels together so as to form a module outer surface;
fixedly connecting an adjustable mount to the module; and
configuring the mount to set an orientation of the module with respect to the ROI and to set a location of the module outer surface with respect to the outer surface of the body so that all of the photon counting detectors are able to simultaneously receive from the ROI the respective flux of the photons.

29. A method for imaging, comprising:
forming a first image of a region of interest (ROI);
identifying a location in the first image of a source of radiation in the ROI;
adjusting positions and orientations of radiation detectors in response to the location; and
operating the radiation detectors to generate a second image of the ROI.

30. The method according to claim 29, and comprising, prior to adjusting the positions and the orientations of the radiation detectors, simulating operation of the radiation detectors to generate a simulated image of the ROI, and wherein adjusting the positions and the orientations of the radiation detectors comprises adjusting the positions and the orientations and acquisition times of the radiation detectors in response to the simulated image.

31. The method according to claim 30, wherein simulating the operation of the radiation detectors comprises implementing scanning strategies comprising detector parameters for the radiation detectors, and generating respective different simulated images comprising the simulated image, in response to the scanning strategies.

32. The method according to claim 31, wherein a given scanning strategy comprises sets of the detection parameters, and wherein each set comprises for the radiation detectors respective positions, respective orientations at the positions, and respective acquisition times at the positions.

33. The method according to claim 31, and comprising selecting an optimal scanning strategy for the radiation detectors in response to the different simulated images, and applying the optimal scanning strategy to the radiation detectors.

34. The method according to claim 33, wherein applying the optimal scanning strategy comprises implementing sets of the detection parameters of the radiation detectors sequentially.

35. The method according to claim 34, and comprising, for a given set of the detection parameters, performing a comparison of results derived from signals received from the radiation detectors with expected results derived from simulated signals for the optimal scanning strategy, and in response to the comparison repeating the given set.

36. The method according to claim 29, and comprising:
simulating operation of the radiation detectors to generate a simulated image of the ROI prior to adjusting the positions and the orientations of the radiation detectors; and
determining parameters of the ROI and storing the parameters in a table providing a correspondence between the parameters of the ROI and the positions and the orientations and acquisition times of the radiation detectors,
wherein adjusting the positions and the orientations of the radiation detectors comprises accessing the table and adjusting the positions and the orientations and the acquisition times in response to the correspondence.

37. The method according to claim 29, wherein adjusting the positions of the radiation detectors comprises arranging the radiation detectors in a free-field-of-view topology wherein no radiation detectors are within a field of view of a given radiation detector.

38. The method according to claim 37, wherein the field of view comprises the ROI.

39. Apparatus for imaging, comprising:
radiation detectors; and
a processor which is configured to:
form a first image of a region of interest (ROI),
identify a location in the first image of a source of radiation in the ROI,
adjust positions and orientations of the radiation detectors in response to the location, and
operate the radiation detectors to generate a second image of the ROI.

40. The apparatus according to claim 39, wherein the processor is configured to, prior to adjusting the positions and the orientations of the radiation detectors, simulate operation of the radiation detectors to generate a simulated image of the ROI, and wherein adjusting the positions and the orientations of the radiation detectors comprises adjusting the positions and the orientations and acquisition times of the radiation detectors in response to the simulated image.
